(19) United States
(12) Patent Application Publication
(10) Pub. No.: US 2024/0091043 A1
(43) Pub. Date: Mar. 21, 2024

(54) FOOTWEAR FOR POSTOPERATIVE PROTECTION AND REHABILITATION

(71) Applicants: Daniel Yiang WU, Hong Kong (HK); Hong Kong Productivity Council, Hong Kong (HK)

(72) Inventors: Daniel Yiang WU, Hong Kong (HK); Ming GE, Hong Kong (HK); Kit Sum Samantha CHAN, Hong Kong (HK); Ho Nam Ernest YIM, Hong Kong (HK)

(21) Appl. No.: 18/520,097

(22) Filed: Nov. 27, 2023

Related U.S. Application Data
(63) Continuation of application No. 17/661,554, filed on Apr. 30, 2022.

(51) Int. Cl. A61F 5/01; A43B 7/1445; A43B 7/1464

(52) U.S. Cl. CPC A61F 5/0195; A43B 7/1445; A43B 7/1464

(57) ABSTRACT

The present application provides a footwear for postoperative protection and rehabilitation. The protective footwear specifically structurally includes: a footwear body; an air bag system, provided at a position of the footwear body corresponding to a human metatarsal region; and a fixing frame, provided at the position of the footwear body corresponding to the human metatarsal region, wrapping a compressed air bag, and comprising a flat plate and two arc-shaped plates extending upward and inward from two sides of the flat plate; where, when a human foot is squeezed by gravity, air in a bottom air reservoir is squeezed into side-flap air reservoirs, such that the side-flap air reservoirs are inflated; and restricted by the fixing frame, the side-flap air reservoirs generate an inward pressure to the metatarsal region to reduce a tensile stress generated between metatarsals when the human foot moves. The footwear in the embodiments of the present application can replace a plaster cast to fix and protect the metatarsal region of the foot, and it is convenient to wear and disassemble without affecting postoperative examination and rehabilitation of the foot.

### CROSS-REFERENCE TO RELATED APPLICATION(S)

This application is a continuation of the U.S. application Ser. No. 17661554 filed on Apr. 30, 2022, and entitled “FOOTWEAR FOR POSTOPERATIVE PROTECTION AND REHABILITATION”.

### TECHNICAL FIELD

The present application relates to the technical field of footwear, and in particular to a footwear for protection and rehabilitation after bunion/hallux valgus corrective surgery.

### BACKGROUND

Bunion or hallux valgus is a common deformity of the foot. It is closely related to the underlying metatarsus primus varus deformity, which is tilting of the first metatarsal bone away from the second metatarsal bone. The migrated distal end of the first metatarsal bone juts out the foot's big toe side to form the bony prominence called the bunion deformity. The big toe would also be tilted in the opposite direction in response to the first metatarsal bone shifting, which creates the foot's hallux valgus deformity. However, the cause of the first metatarsal bone displacement is its stabilizing ligaments' incompetence and failure.

These bones' misalignment is progressive and eventually will cause pain and compromise the foot's mechanical function in walking, increasing difficulties and thus reluctance to walk. There are, unfortunately, no effective ways to correct or stop the progression of this condition without surgery when it becomes necessary.

The surgery is to realign the displaced bones and create a new fibrous connecting bridge between the first and second metatarsals to stop the first metatarsal bone separating from the second metatarsal bone again in the future. The first metatarsal realignment is achieved by tying it to the second metatarsal bone and maintained temporarily only by the holding sutures' limited strength. Besides, the fibrous connective bridge will take 4 months to become strong enough for unprotected walking. Currently, during these first few months of waiting for the new connection to form after surgery, a foot cast is applied to protect the sutures from loosening or breaking under stress in walking. But, casts are difficult and time-consuming, and costly to apply well. They are not user-friendly for prolonged usage. It will also be less protective when it becomes loose as postoperative swellings subside.

### SUMMARY

Embodiments of the present application provide a footwear for postoperative protection and rehabilitation to solve most if not all shortcomings of the prior art.

This protective footwear consists of three main parts for different purposes.

A first part is an air bag system serving as a dynamic air-compression system confined in an unyielding rigid frame. The air bag system is partly filled and has a bottom air reservoir and side-flap reservoirs connected to two sides of the bottom air reservoir. The purpose of the air bag system is to provide an alternating protecting and resting cycle in walking. In walking, each human foot goes through a repeating stance-swing cycle. A stance phase is when the entire body is standing on one foot while the other foot is off the ground moving forward in the air during its swing phase. The embodiments of the present application can provide protection for the foot during the stance phase and rest the foot during the swing phase. The separation tension force between the first and second metatarsals of the forefoot can be minimized during the protecting phase and free of constant pressure during the resting phase. The air bag system allows air to be forced from the bottom air reservoir during the stance phase to the side-flap air reservoirs. As the side-flap air reservoirs are inflated, they provide side compression forces on the forefoot to reduce tensile stress on the holding sutures between the first and second metatarsals.

A second part is a footwear body, including an inner footwear body and an outer sole, which are detachably connected.

The inner footwear body includes an inner sole and a casing fixed to the inner sole. The casing includes at least two side vamps. The inner footwear body provides a rigid sole to restrict the foot from the normal rolling movement in walking.

The outer sole includes an outer sole body and a toe cap fixed on the outer sole body. The outer sole is detachably provided below the inner sole. The outer sole can be used for outdoor usage. The footwear can be conveniently removed for early wound care, daily foot hygiene and resting indoors. The toe cap is used for toe protection.

A third part is a rigid and unpliable fixing frame with a C-shaped section. The fixing frame has a rigid quality that directs the outwardly expanding air at the side flaps to compress inward. During the swing phase, they are deflated as air returns to the bottom air reservoir when all weight is lifted off it. The forefoot will then be free of any lateral compression pressure. This on-and-off pressure cycle can not only provide much improved foot protection to allow an early active and safe postoperative life but also massage therapy to help promote circulation and rid of postoperative swellings.

The subject matter of the embodiments of the present application as described above can replace a plaster cast to fix and protect the metatarsal region of the foot, and it is convenient to wear and disassemble without affecting postoperative examination and rehabilitation of the foot. The protective footwear of the present application can be used to protect the foot in rehabilitation after bunion/hallux valgus corrective surgery, as well as to protect the ankle and other parts of the foot after other injuries and operations with this dynamic air compression system.

The above summary is for the purpose of the description only and is not intended to be limiting in any way. Further aspects, embodiments, and features of the present application will be readily apparent from reference to the drawings and the following detailed description, in addition to the illustrative aspects, embodiments, and features described above.

### BRIEF DESCRIPTION OF THE DRAWINGS

In the drawings, the same reference numerals refer to like or similar components or elements throughout the multiple drawings unless otherwise specified. The drawings are not necessarily drawn to scale. It should be understood that the drawings depict only some embodiments of the present application and should not be construed as limiting the scope of the present application.

FIG. 1 is a perspective view of an outer case of protective footwear according to an embodiment of the present application;

FIG. 2A is another perspective view of the outer case of the protective footwear according to an embodiment of the present application;

FIG. 2B is an exploded view of the outer case of the protective footwear according to an embodiment of the present application;

FIG. 3 is a top view of the outer case of the protective footwear according to an embodiment of the present application;

FIG. 4 is a bottom view of the outer case of the protective footwear according to an embodiment of the present application;

FIG. 5 is a perspective view of a compressed air bag in a flattened state according to an embodiment of the present application;

FIG. 6 is a front view of the compressed air bag in a flattened state according to an embodiment of the present application;

FIG. 7 is a front view of the compressed air bag in a back-flattened state according to an embodiment of the present application;

FIG. 8 is a perspective view of the compressed air bag in a flattened state according to an embodiment of the present application;

FIG. 9 is a perspective view of an outer sole according to an embodiment of the present application;

FIG. 10 is a perspective view of a fixing frame according to an embodiment of the present application;

FIG. 11 is a side view of the protective footwear with the casing according to an embodiment of the present application;

FIG. 12 is a perspective view of the compressed air bag according to an embodiment of the present application;

FIG. 13 is a perspective view of a toe separation piece fixed on the protective footwear according to an embodiment of the present application;

FIG. 14 is a perspective view of a fixing frame according to an embodiment of the present application;

FIG. 15 is a perspective view of the toe separation piece according to an embodiment of the present application; and

FIG. 16 is a perspective view of a fixing member according to an embodiment of the present application.

### DETAILED DESCRIPTION OF THE EMBODIMENTS

Hereinafter, only certain exemplary embodiments are briefly described. As will be appreciated by those skilled in the art, the described embodiments may be modified in a variety of different ways without departing from the spirit or scope of the present application. Accordingly, the figures and description are considered to be exemplary in nature and not limiting.

The embodiments of the present application provide an interlocking mechanism such that an outer sole of protective footwear can be detachably provided on an inner sole, which is convenient for patients to wear the protective footwear in two different environments, indoor and outdoor. Since the protective footwear is detachable, compared to a plaster cast, it is also convenient to clean the foot. Indoors, the outer sole can be detached from the inner sole, and the foot can be more comfortable and still protected. Outdoors, the outer sole can be provided on the inner sole to protect the foot from the impact of outdoor hard objects. In addition, the embodiments of the present application can be applied to the protective footwear for mountain hiking, cross-country walking, skiing, or any foot and ankle support/protection purposes.

The detachable structure of the protective footwear will be described in detail below:

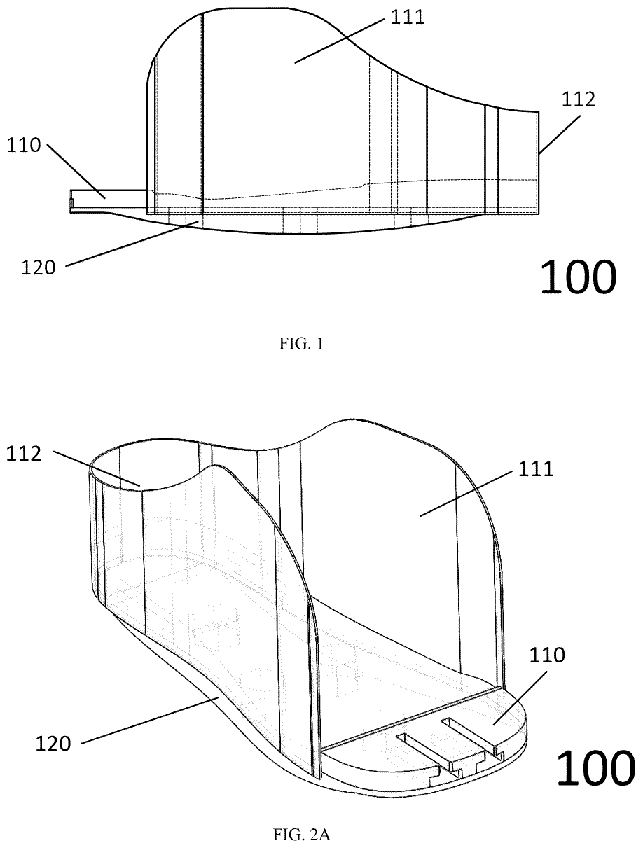

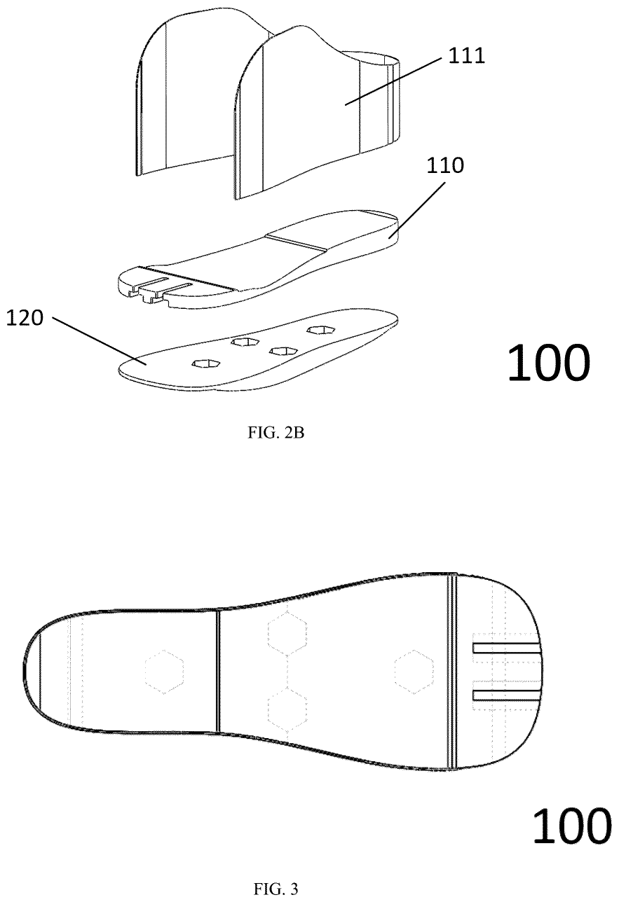

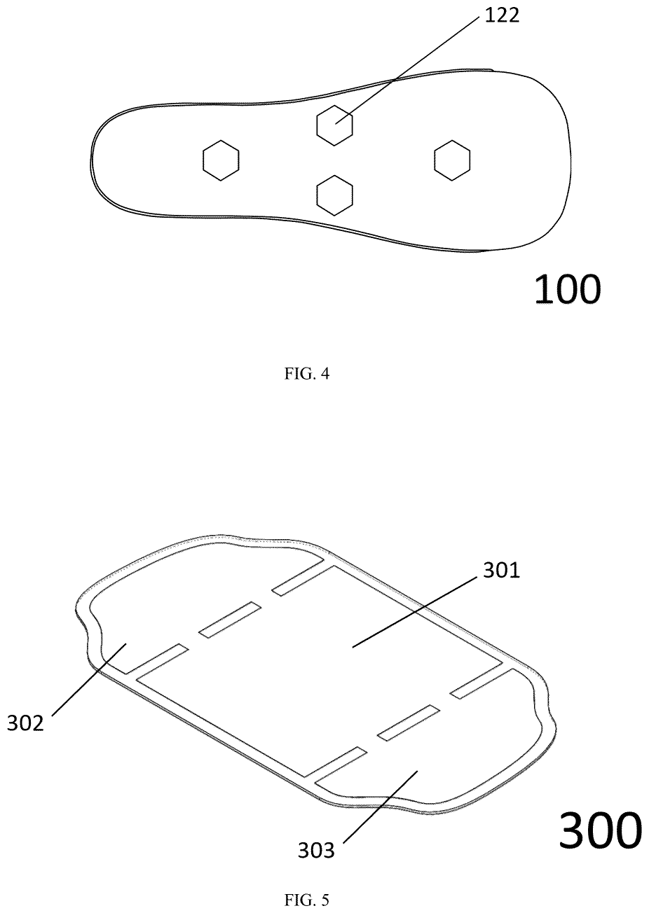

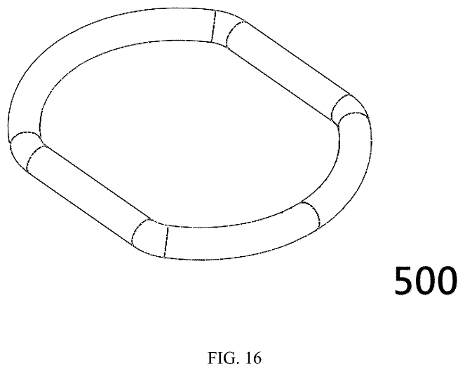

Please refer to FIGS. 1 to 16, in which every schematic view of every component of this footwear for postoperative protection and rehabilitation is shown. The protective footwear includes an outer case 100, a compressed air bag 300 and an outer sole 140 etc. As shown in FIG. 1, the outer case 100 includes an inner sole 120, and a casing 110, a fixing frame 900 and a toe separation piece 400 fixed on the inner sole 120. The casing 110 includes at least two side vamps 111. In some embodiments, the casing further includes a heel 112. The two side vamps 111 may move toward the instep and partially wrap the foot by using a Velcro. In addition, a curved bottom of the inner sole 120 is convenient for the patient to walk.

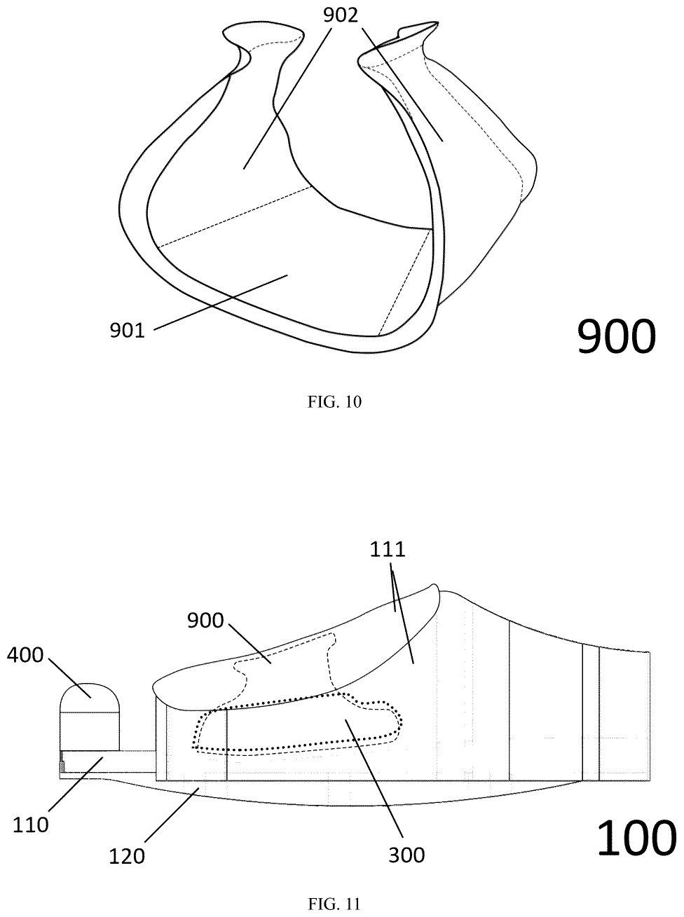

As shown in FIG. 9, the outer sole 140 may include an outer sole body 142 and a toe cap 141 fixed on the outer sole body 142, and the outer sole 140 is detachably provided below the inner sole 120.

The footwear provided in the embodiments of the present application is detachable, which facilitates walking indoors and outdoors. When the user walks outdoors, the outer sole 140 is fixed on the inner sole 120. When the user walks indoors, the outer sole 140 is removed from the inner sole 120. After the user removes the outer sole 140 from the inner sole 120 indoors, since the toe cap 141 is provided on the outer sole 140, the toe cap 141 is also removed from the inner sole 120. Hence, the toes of the foot can stretch when the user takes exercises indoors to relax the foot. On the other hand, when walking outdoors, the user fixedly attaches the outer sole 140 to the inner sole 120 and the toe cap 141 is also fixedly attached to the footwear, such that the toes are protected to avoid injuries outdoors.

The detachable connection structure between the inner sole and the outer sole can be designed as follows:

In some embodiments, multiple openings 122 are provided on the inner sole 120, and multiple screw bolts 124 matched with the multiple openings 122 are provided on the outer sole 140. A minimum diameter of the multiple openings 122 is not less than a shank diameter of the screw rods 124, and a maximum diameter of the multiple openings 122 is less than a head diameter of the screw bolts 124. The outer sole 140 is fitted into the inner sole 120. Then the multiple screw bolts 124 are correspondingly screwed into the multiple openings 122 on the inner sole 120, such that heads of the screw bolts 124 are fastened on the openings 122, thereby fixing the inner sole 120 onto the outer sole 140.

In some embodiments, two side flaps of the outer sole 140 close to the metatarsal are provided with at least a pair of openings 143. The protective footwear further includes at least a fixing belt that may pass through the openings 143 to fix the entire outer case 100 (including the inner sole 120 and the casing 110 fixed on the inner sole 120) onto the outer sole 140. The embodiments of the present application can further strengthen the connection of the inner sole 120 and the outer sole 140.

In the above embodiment, a compressed air bag 300 can be used to protect the metatarsal region of the foot and facilitate walking indoors and outdoors. The compressed air bag is located in the metatarsal component of the protective footwear, and is composed of two parts, a foot pad portion 301 and two side flaps 302, 303 extending from two sides of the foot pad portion 301. The shape of the compressed air bag is similar to a traditional plaster cast. The inside of the compressed air bag is filled with air that can flow and can be deformed into different shapes, making the compressed air bag tightly and firmly wrap the healing part of the foot. When the patient puts on the protective footwear and performs exercises such as walking, standing or resting, the air in the compressed air bag in the protective footwear moves freely with the squeezing foot, which can provide support and protection for the foot and fix the metatarsal region of the foot.

The compressed air bag 300 is located at the metatarsal region of the casing 110 and the compressed air bag 300 is full of air for protecting the metatarsal component of the foot. As shown in FIGS. 5 and 7, which represent a flattened view of the compressed air bag 300, the compressed air bag 300 includes a foot pad portion (a bottom air reservoir) 301 covering the metatarsal region of the casing 110 and two side flaps 302, 303 protruding laterally from two sides of the foot pad portion 301. The two side flap portions 302, 303 may bend and fold from the two sides of the foot pad portion 301 toward the foot. Thus, the entire compressed air bag 300 may wrap the metatarsal region of the foot, and after the outer case 100 is taken off, the compressed air bag 300 may be opened easily to release the foot, so as to prevent the foot from being pressured.

The structure of the compressed air bag will be explained in detail as follows:

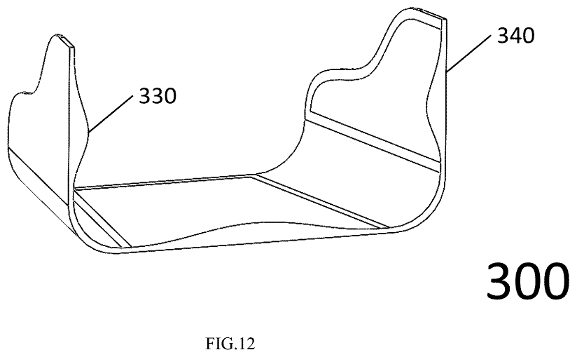

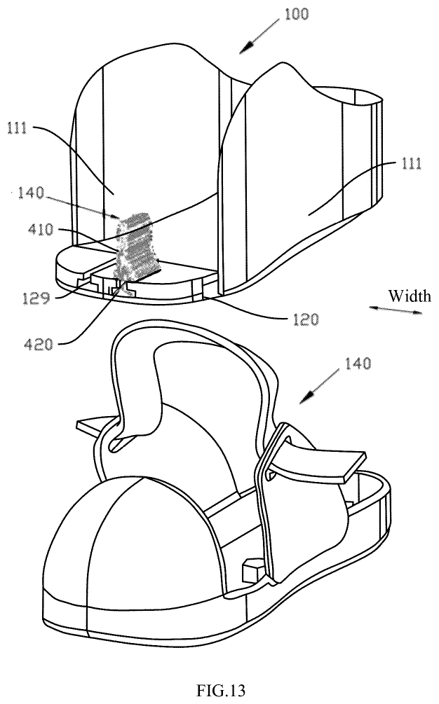

As shown in FIGS. 5 and 12, the foot pad portion 301 of the compressed air bag 300 has a flattened shape, with a surface (an inner protective layer 330 of the compressed air bag) close to the foot (contacts the foot) and a surface (an outer layer 340 of the compressed air bag) close to the casing 110 (contacts the casing 110). On the foot pad portion, the cross-section of the outer layer 340 of the compressed air bag may have a half-arc shape on the side flaps. In the embodiment of the present application, when the foot pad portion is compressed, the air in the foot pad portion flows to the side flaps, thereby providing a lateral pressure for the foot. As shown in FIG. 7, the outer layer of the compressed air bag 330 is flat.

When the compressed air bag 300 is wrapped up to wrap the foot, the cross-section may be that shown in FIG. 12. Inside the compressed air bag 300, three spaced-apart air-filled air reservoirs are provided. The air reservoirs may be fixed on an inner surface of the compressed air bag 300 close to the foot.

In some embodiments, the outer layer of the compressed air bag 300 is provided with a Velcro. When the user bends and folds the two side flaps 302, 303 of the compressed air bag 300 together toward the instep, the compressed air bag is fixed inside the fixing frame by the Velcro.

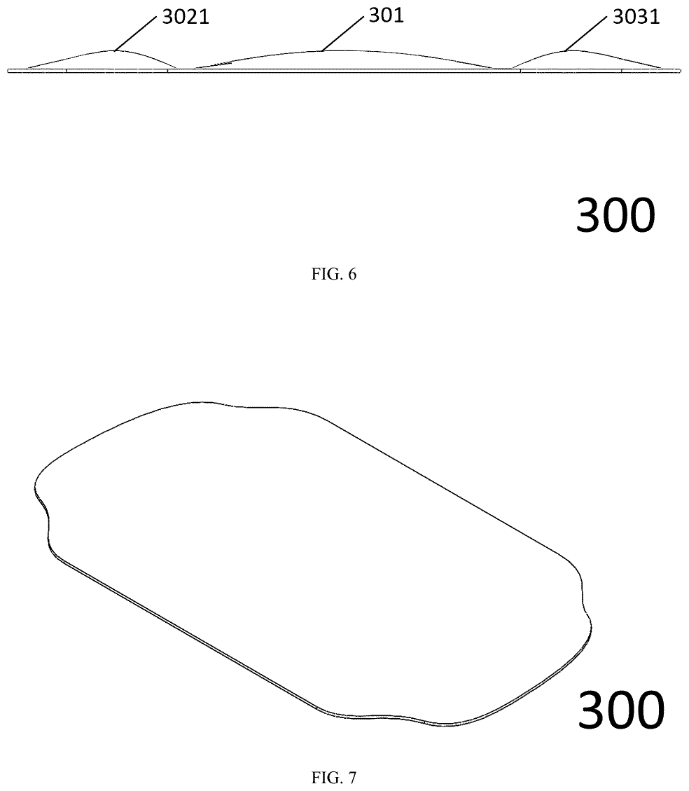

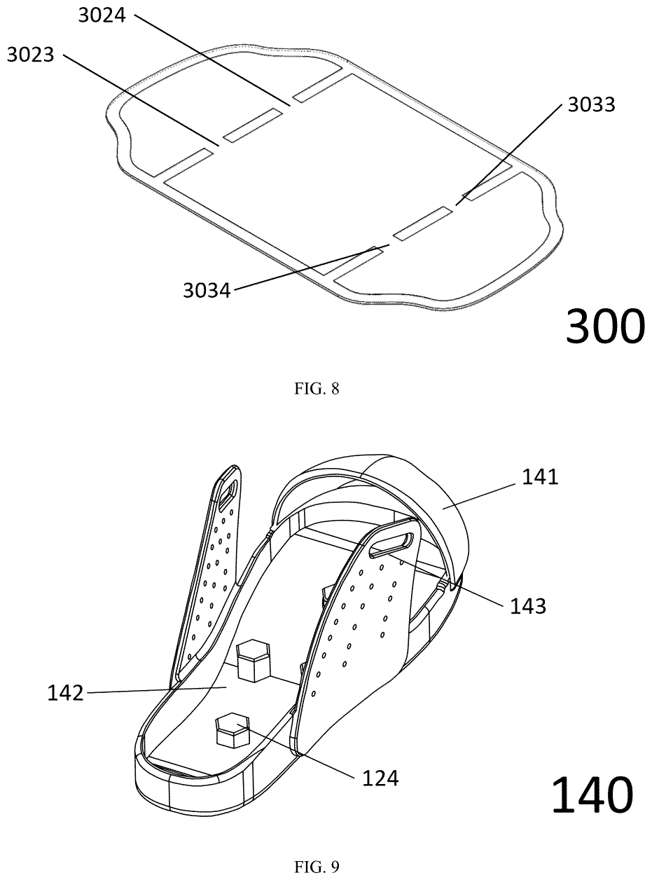

For example, as shown in FIG. 6, the side flaps 302, 303 respectively include side-flap air reservoir 3021, 3031, and one side of each of the side-flap air reservoirs 3021, 3031 is connected to the foot pad portion 301 serving as the bottom air reservoir. As shown in FIG. 8, the side-flap air reservoirs 3021, 3031 are connected to the bottom air reservoir 301 through air flow channels 3023, 3024, 3033, 3034.

As shown in FIG. 10, the protective footwear further includes a fixing frame 900 for fixing the compressed air bag. The fixing frame 900 includes a flat plate 901 and two arc-shaped plates 902 extending from two sides of the flat plate 901 upward and inward. An outer circumferential shape of the flat plate 901 is similar to or the same as that of the foot pad portion of the compressed air bag 300. When the two arc-shaped plates 902 are unfolded, the outer circumferential shape of the two arc-shaped plates 902 is similar to or the same as that of the two side flaps of the compressed air bag 300. The fixing frame 900 can fix the compressed air bag 300 in a folded state to wrap the foot.

Referring to FIG. 14, further, in order to prevent the fixing frame 900 from rebounding, in other embodiments, ends of the arc-shaped plates 902 are bent to form hook portions 909. After the two arc-shaped plates 902 are brought closer together, a fixing member 500 is sleeved on the hook portions 909 of the two arc-shaped plates 902 to prevent the fixing frame 900 from rebounding, such that the overall shape of the fixing frame 900 is stable. The fixing member 500 refers to a member that can be sleeved on the hook portions 909, which can be an elastic band, a fixing ring, etc., and more forms of the fixing member will not be exemplified herein.

Further, the ends of the arc-shaped plates 902 are folded inwardly to form a bent area with a width smaller than that of a middle portion, so as to form the hook portions 909. When the two arc-shaped plates 902 are brought closer together, the hook portions 909 are located in an area enclosed by the fixing frame 900. Specifically, the width of the bent area of the arc-shaped plates 902 is smaller than that in the middle of the arc-shaped plates 902 so as to reduce the size of the fixing member 500. In this way, the protective footwear of this embodiment is more compact (the drawings of this embodiment are not illustrated for the time being).

It should be understood that the width direction indicated in FIG. 14 is for the convenience of understanding the shape of the fixing frame 900, and is not intended to limit the relevant terms of the present application.

In some embodiments, double-sided Velcro is attached to the two side vamps 111. When the user's foot is sheathed in the compressed air bag 300, the compressed air bag is fixed inside the fixing frame by the Velcro. The two side flaps 302, 303 of the compressed air bag 300 are bent with the sides of the fixing frame, and the fixing frame is fixed to the casing 110 by the Velcro. Then, through the double-sided Velcro on the two side vamps 111, the instep is completely wrapped, and the entire outer case is fixed.

The structure of the toe separation piece 400 will be described below.

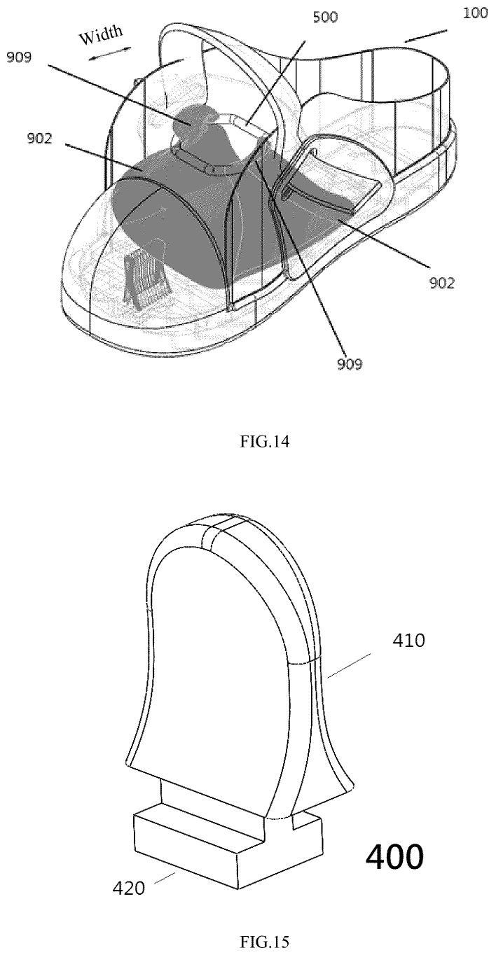

Referring to FIG. 13, the toe separation piece 400 is detachably provided on the inner sole 120. The toe separation piece 400 is located at a position of the inner sole 120 corresponding to a gap between any two toes of the same foot. The toe separation piece 400 is matched with the gap between any two toes of the same foot in terms of shape. The toe separation piece 400 can fix the position of the toes and prevent the toes from moving freely, so as to promote the recovery of the diseased part.

Specifically, the toe separation piece 400 includes a main body and a connecting portion 420 provided at one end of the main body. Two opposite sides of the main body are concavely arranged to form a profiling portion 410 that matches the shape of the side surface of the toe. An end surface of the inner sole 120 extends toward the interior of the inner sole 120 to form an insertion port. The shape of the insertion port is matched with the shape of the connecting portion 420.

More specifically, the width of the connecting portion increases gradually/gradiently in a direction away from the main body, such that the toe separation piece 400 is firmly clamped on the inner sole.

It should be understood that the width direction indicated in FIG. 13 is for the convenience of understanding the shape of the toe separation piece 400, and is not intended to limit the relevant terms of the present application.

The following will describe another embodiment of protective footwear, specifically as follows:

As an exemplary embodiment, FIG. 11 is a side view of the protective footwear with a casing according to an embodiment of the present application. As shown in FIG. 11, the footwear according to an embodiment of the present application may include an outer case 100 and a compressed air bag 300. The outer case 100 may include a casing 110 and an inner sole 120. The casing 110 includes at least two side vamps 111 and a heel 112. The casing 110 is detachably provided on the inner sole 120 to protect the foot and compressed air bag 300. When the side vamps 111 bend toward the instep, two opposite sides of the two side vamps 111 are overlapped and attached through a Velcro.

As shown in FIG. 11, the casing 110 does not include the toe cap so as to ensure ventilation. As the outer sole is not attached to the inner sole 120, the inner sole 120 may be used as an outsole, making it convenient for protected indoor walking.

As shown in 12, the compressed air bag 300 includes an inner protective layer 330 and an outer layer 340 that are connected together at edge portions to form the air bag. The inner protective layer 330 is close to the foot and the outer layer 340 is away from the foot.

In the embodiment of the present, when the foot pad portion is compressed, the air in the foot pad portion flows to the side flaps, thereby providing a lateral pressure for the foot. The two side flaps of the compressed air bag can be deformed into different shapes to tightly and securely wrap the healing part of the foot. The dynamic air flow in the compressed air bag 300 provides support for free movement and rest of the foot of the patient. In addition, it also provides protection and rigid support. Before the compressed air bag 300 is applied to the protective footwear, the compressed air bag 300 may be subjected to a flow simulation to verify the compressed air flow and check the tensile stress of key sealing areas.

As an exemplary embodiment, the sole according to the embodiment of the present application may include two soles, namely the inner sole 120 and the outer sole 140. The inner sole 120 is used as an indoor sole for indoor use. The outer sole 140 is used as an outdoor sole. When the user walks outdoors, the user may fit the outdoor sole under the indoor sole to protect the foot. In order that it is convenient for the foot to breathe indoors, the casing 110 does not include the toe cap 141. However, when the user goes outdoors, for the sake that the toes are protected from undue injuries, the toe cap 141 is required to protect the toes. For this purpose, the outer sole 140 is additionally provided with the toe cap 141 that couples the casing 110. The outer sole 140 and the inner sole 120 are detachably connected, and the toe cap 141 of the outer sole 140 is also detachably attached to the footwear. When the user walks indoors, the user may remove the outer sole 140 from the footwear. When the user walks indoors to outdoors, the user may fit the outer sole 140 below the inner sole 120, such that the toe cap 141 is fixed at the toe part.

The embodiments of the present application provide protective footwear, which is designed for purpose of medical rehabilitation. Firstly, the compressed air bag 300 has a unique design, which allows dynamic air flows inside, such that the foot can provide rigid support to the healing area during different movements of the foot. Secondly, the detachable footwear structure has high flexibility in different environmental applications, e.g. indoors or outdoors.

Compared with the prior art, the present application has the following beneficial effects.

The compressed air bag 300 according to the present application provides reliable support and protection to the healing site of the metatarsal of the foot, which allows the healing site to recover healthily and correctly. Furthermore, the outer case 100 is provided to protect the compressed air bag 300, and the outer case 100 is detachable. In addition, a structure of an indoor sole and an outdoor sole is provided, which allows the outdoor sole of the footwear to be detached indoors, making it convenient for the patient to walk indoors or for a medical examination. When walking outdoors, the user may attach the outdoor sole to the footwear. Compared with the existing plaster cast, when a medical examination is needed, some parts of the detachable footwear according to the present application may be reused to reduce the cost.

The structures of the detachable outer case 100 and the compressed air bag 300 according to the present application are only exemplary embodiments, which may be widely applied in medical rehabilitation products, or other mechanical solutions related to the air bag. For example, the present application is suitable for rehabilitation treatment. Additionally to the above described nature, the present application is more advantageous than a normal support structure. The present application may be applied in other industries, such as protective wearing, safety application for vehicle and equipment for extreme sports competition. In addition, the concept of the detachable footwear structure may motivate the development of multi-usage shoes under all environmental conditions. For example, the shoemaking industry can design and provide different additional outer soles or sub-elements that fulfill their purpose.

In addition, the protective footwear according to the present application is convenient to clean due to its detachable design and the design of the indoor sole and the outdoor sole. More importantly, the present application allows the foot to be examined during rehabilitation, which is not allowed in a normal plaster cast, unless that the plaster cast is removed. The removed plaster cast cannot be double-used and a new plaster cast must be made. Furthermore, based on the structure of the indoor sole and the outdoor sole according to the present application, the present application may be designed as a mountain shoe, ski shoe, safety shoe or non-slippery shoe etc.

It should be noted that, in the description of the present application, the terms such as “central”, “longitudinal”, “transverse”, “long”, “wide”, “thick”, “upper”, “lower”, “front”, “back”, “left”, “right”, “vertical”, “horizontal”, “top”, “bottom”, “inner”, “outer”, “clockwise”, “anticlockwise”, “axial”, “radial” and “circumferential” are used to indicate orientations shown in the drawings. It should be noted that these terms are merely intended to facilitate a simple description of the present application, rather than to indicate or imply that the mentioned apparatus or elements must have the specific orientation or be constructed and operated in the specific orientation. Therefore, these terms may not be construed as a limitation to the present application.

Moreover, the terms such as “first” and “second” are used only for the purpose of description and should not be construed as indicating or implying a relative importance, or implicitly indicating a quantity of indicated technical features. Thus, features defined with “first” and “second” may explicitly or implicitly include one or more of the features. In the description of the present application, “multiple” means two or more, unless otherwise specifically defined.

In the present application, unless otherwise clearly specified, the terms “installation”, “interconnection”, “connection” and “fixation” are intended to be understood in a broad sense. For example, the connection may be a fixed connection, removable connection or integral connection; may be a mechanical connection or electrical connection; may be a direct connection or indirect connection using a medium; and may be a communication or interaction between two elements. Those of ordinary skill in the art should understand the specific meanings of the above terms in the present disclosure based on specific situations.

In the present application, unless otherwise expressly specified and defined, that a first feature is “above” or “under” a second feature may include that the first feature is in direct contact with the second feature, or that the first feature and the second feature are not in direct contact with each other but are in contact by using another feature between them. In addition, that the first feature is “over”, “above”, and “on” the second feature includes that the first feature is directly above and diagonally above the second feature, or simply indicates that a horizontal height of the first feature is larger than that of the second feature. The case where the first feature is “beneath”, “below”, and “under” the second feature includes that the first feature is directly below and diagonally below the second feature, or simply indicates that the altitude of the first feature is lower than that of the second feature.

The disclosure herein provides multiple different embodiments or examples to implement different structures of the present application. To simplify the disclosure of the present application, components and dispositions of particular examples are described herein. Certainly, the descriptions are only examples and are not intended to limit the present application. In addition, reference numerals and/or reference letters may be repeated in different examples in the present application, and such repetition is for purposes of simplification and clarity and is not indicative of relationships between the embodiments and/or dispositions discussed.

The above merely describes specific implementations of the present application, but the protection scope of the present application is not limited thereto. Any person skilled in the art can easily conceive modifications or replacements within the technical scope of the present application, and these modifications or replacements shall fall within the protection scope of the present application. Therefore, the protection scope of the present application should be subject to the protection scope of the claims.

What is claimed is:

1. A footwear for postoperative protection and rehabilitation, comprising:
a footwear body;
an air bag system, provided at a position of the footwear body corresponding to a metatarsal region of a human foot; and
a fixing frame, provided at the position of the footwear body corresponding to the metatarsal region of the human foot, wrapping a compressed air bag, and comprising a flat plate and two arc-shaped plates extending upward and inward from two sides of the flat plate; and
wherein, when the human foot is squeezed by gravity, air in a bottom air reservoir enters side-flap air reservoirs, such that the side-flap air reservoirs are inflated; and restricted by the fixing frame, the side-flap air reservoirs generate an inward pressure to the metatarsal region to reduce a tensile stress between metatarsals generated when the human foot moves.

2. The footwear according to claim 1, wherein the air bag system comprises the compressed air bag, which is configured to wrap the metatarsal region of the human foot, and comprises the bottom air reservoir and the two side-flap air reservoirs extending from two sides of the bottom air reservoir to wrap two side flaps of the metatarsal region of the human foot; and the two side-flap air reservoirs are communicated with the bottom air reservoir through an air flow channel; and
wherein, when the human foot walks wearing the air bag system, the human foot squeezes the bottom air reservoir; the air in the bottom air reservoir enters the side-flap air reservoirs, such that the side-flap air reservoirs are inflated to reduce the tensile stress between the metatarsals generated when the human foot moves, thereby protecting the metatarsal region of the human foot.

3. The footwear according to claim 1, the footwear body comprises:
an inner footwear body, comprising an inner sole and a casing that is fixed on the inner sole and at least comprises two side vamps; and
an outer sole, detachably provided below the inner sole, and comprising an outer sole body and a toe cap fixed on the outer sole body.

4. The footwear according to claim 1, further comprising:
a toe separation piece, detachably provided at a position of the inner sole corresponding to a gap between any two toes of the same foot, and matched with the gap between any two toes of the same foot in terms of shape.

5. The footwear according to claim 4, wherein the toe separation piece is suitable for left and right feet by offsetting upper and lower parts of the toe separation piece to form two different toe separation positions, and suitable for most foot shapes.

6. The footwear according to claim 2, wherein
the fixing frame is provided at a position of the footwear body corresponding to the metatarsal region of the human foot, configured to wrap the air bag system, and comprising a flat plate located on the bottom air reservoir and two arc-shaped plates extending upward and inward from two sides of the flat plate; and
wherein, when the human foot wearing the footwear is squeezed by gravity, the air in the bottom air reservoir of the air bag system is squeezed into the side-flap air reservoirs of the air bag system, such that the side-flap air reservoirs are inflated; and restricted by the fixing frame, the side-flap air reservoirs generate an inward pressure to the metatarsal region of the human foot, so as to reduce the tensile stress between the metatarsals of the human foot generated when the human foot moves.

7. The footwear according to claim 1, wherein ends of the arc-shaped plates are at least partially bent to form hook portions.

8. The footwear according to claim 1, wherein the ends of the arc-shaped plates are bent outward.

9. The footwear according to claim 8, further comprising a fixing ring sleeved on the hook portions and configured to stabilize the fixing frame so as to prevent the fixing frame from expanding outward under a pressure of the air reservoirs.

10. The footwear according to claim 1, wherein the inner footwear body further comprises an insole.
